(19) United States
(12) Patent Application Publication
(10) Pub. No.: US 2017/0245743 A1
(43) Pub. Date: Aug. 31, 2017

(54) ENDOSCOPIC SYSTEM AND ENDOSCOPE

(71) Applicant: OLYMPUS CORPORATION, Tokyo (JP)

(72) Inventor: Hideharu MIYAHARA, Kamiina-gun (JP)

(73) Assignee: OLYMPUS CORPORATION, Tokyo (JP)

(21) Appl. No.: 15/594,714

(22) Filed: May 15, 2017

Related U.S. Application Data
(63) Continuation of application No. PCT/JP2015/082724, filed on Nov. 20, 2015.

(30) Foreign Application Priority Data
Nov. 20, 2014 (JP) PCT/JP2014/080778

(51) Int. Cl. A61B 1/045; A61B 1/00; H04N 5/225; A61B 1/005

(52) U.S. Cl. CPC A61B 1/045; A61B 1/005; A61B 1/00117; H04N 5/2256; H04N 2005/2255

(57) ABSTRACT

An endoscopic system includes: an endoscope acquiring an image of an inside of a subject; and a processing device performing image processing on the acquired image. The endoscope includes: an imaging unit outputting the image of the inside of the subject as an electrical signal; and an optical transmission unit converting the electrical signal into an optical signal and transmitting the optical signal to the processing device via an optical fiber. The processing device includes: an optical reception unit receiving the optical signal transmitted from the optical transmission unit and converts the received optical signal into an electrical signal; a curvature-detecting unit detecting a curvature of the optical fiber, and a control unit controlling at least one of characteristics of the electrical signal output from the imaging unit and characteristics of the electrical signal output from the optical transmission unit based on the detection result of the curvature-detecting unit.

### CROSS REFERENCE TO RELATED APPLICATIONS

This application is a continuation application based on a PCT Patent Application No. PCT/JP2015/082724, filed Nov. 20, 2015, whose priority is claimed on a PCT Patent Application No. PCT/JP2014/080778, filed Nov. 20, 2014, the contents of which are incorporated herein by reference.

### BACKGROUND OF THE INVENTION

Field of the Invention

The present invention relates to an endoscopic system and an endoscope.

Description of the Related Art

An endoscopic system is conventionally used to observe an organ of a subject such as a patient in the field of medicine. The endoscopic system includes an imaging device (an electronic scope) that has, for example, a flexible long and thin shape and is inserted into a body cavity of a patient, an imaging element that is disposed at a tip of the imaging device to capture an internal image, a processing device (an external processor) that performs predetermined image processing on the internal image captured by the imaging element, and a display device that displays the internal image subjected to the image processing by the processing device. When an internal image is captured using the endoscopic system, an insertion section is inserted into a body cavity of a subject, a biological tissue in the body cavity is irradiated with illumination light from a tip of the insertion section, and the imaging element captures an internal image. An operator such as a doctor observes an organ of the subject based on the internal image which is displayed by the display device.

As such an endoscopic system, for example, Japanese Unexamined Patent Application, First Publication No. 2013-192796 discloses a technique of outputting internal image information captured by an imaging element as an optical signal to a processing device via an optical fiber using a light-emitting element disposed in an imaging device. In this technique, transmission output characteristics of the light-emitting element are controlled to appropriately transmit internal image information even when an output level of the light-emitting element decreases due to an ambient temperature of the imaging device.

### SUMMARY OF THE INVENTION

According to a first aspect of the present invention, an endoscopic system is provided, including: an endoscope that acquires an image of an inside of a subject; and a processing device that performs image processing on the acquired image, wherein the endoscope includes an imaging unit that outputs the image of the inside of the subject as an electrical signal and an optical transmission unit that converts the electrical signal into an optical signal and transmits the optical signal to the processing device via an optical fiber, the processing device including an optical reception unit that receives the optical signal transmitted from the optical transmission unit and converts the received optical signal into an electrical signal, a curvature-detecting unit that detects a curvature of the optical fiber, and a control unit that controls characteristics of the electrical signal output from the imaging unit based on the detection result of the curvature-detecting unit, the imaging unit including an amplifier that amplifies the electrical signal, and the control unit controlling the characteristics of the electrical signal output from the imaging unit by changing an amplification factor of the amplifier.

According to a second aspect of the present invention, in the endoscopic system according to the first aspect, the curvature-detecting unit may detect the curvature of the optical fiber based on the optical signal received by the optical reception unit.

According to a third aspect of the present invention, in the endoscopic system according to the second aspect, the curvature-detecting unit may detect the curvature of the optical fiber based on an amplitude of the optical signal received by the optical reception unit.

According to a fourth aspect of the present invention, in the endoscopic system according to the second aspect, the curvature-detecting unit may convert the optical signal received by the optical reception unit into an electrical signal and may detect the curvature of the optical fiber based on the converted electrical signal.

According to a fifth aspect of the present invention, in the endoscopic system according to the first aspect, the control unit may detect an imaging frame rate of the imaging unit and may change an adjustment ratio of the characteristics of the electrical signal output from the imaging unit and characteristics of the optical signal output from the optical transmission unit based on the imaging frame rate.

According to a sixth aspect of the present invention, in the endoscopic system according to the fifth aspect, the control unit may stop controlling the characteristics of the electrical signal output from the imaging unit and may control the characteristics of the optical signal output from the optical transmission unit when the imaging frame rate is equal to or greater than a predetermined value.

According to a seventh aspect of the present invention, in the endoscopic system according to the first aspect, the imaging unit may output a predetermined electrical signal other than the electrical signal of the image of the inside of the subject in a horizontal blanking period or a vertical blanking period, and the curvature-detecting unit may detect the curvature of the optical fiber based on an amplitude of the predetermined electrical signal.

According to an eighth aspect of the present invention, an endoscopic system is provided, including: an endoscope that acquires an image of an inside of a subject; and a processing device that performs image processing on the acquired image, wherein the endoscope includes an imaging unit that outputs the image of the inside of the subject as an electrical signal, an optical transmission unit that converts the electrical signal into an optical signal and transmits the optical signal to the processing device via an optical fiber, and a curvature-detecting unit that detects a curvature of the optical fiber, the processing device including an optical reception unit that receives the optical signal transmitted from the optical transmission unit and converts the received optical signal into an electrical signal and a control unit that controls at least one of characteristics of the electrical signal output from the imaging unit and characteristics of the optical signal output from the optical transmission unit based on the detection result of the curvature-detecting unit, the imaging unit including an amplifier that amplifies the electrical signal, and the control unit controlling the characteristics of the electrical signal output from the imaging unit by changing an amplification factor of the amplifier.

According to a ninth aspect of the present invention, an endoscope is provided, including: an imaging unit that outputs an image of an inside of a subject as an electrical signal; an optical transmission unit that converts the electrical signal into an optical signal and transmits the optical signal to the outside via an optical fiber; and a signal-receiving unit that receives a control signal on characteristics of the electrical signal output from the imaging unit based on a curvature of the optical fiber, wherein the imaging unit includes an amplifier that amplifies the electrical signal, and the amplifier adjusts the characteristics of the electrical signal output from the imaging unit by changing an amplification factor thereof based on the control signal.

### BRIEF DESCRIPTION OF THE DRAWINGS

FIG. 1 is a diagram schematically illustrating a configuration of an endoscopic system according to a first embodiment of the present invention.

FIG. 2 is a block diagram illustrating functional configurations of principal parts of the endoscopic system according to the first embodiment of the present invention.

FIG. 3 is a timing chart illustrating operations of the endoscopic system according to the first embodiment of the present invention.

FIG. 4 is a block diagram illustrating functional configurations of principal parts of an endoscopic system according to a modified example of the first embodiment of the present invention.

FIG. 5 is a timing chart illustrating operations of the endoscopic system according to the modified example of the first embodiment of the present invention.

FIG. 6 is a block diagram illustrating functional configurations of principal parts of an endoscopic system according to a second embodiment of the present invention.

FIG. 7 is a timing chart illustrating operations of the endoscopic system according to the second embodiment of the present invention.

FIG. 8 is a timing chart illustrating operations of an endoscopic system according to a third embodiment of the present invention.

FIG. 9 is a block diagram illustrating functional configurations of principal parts of an endoscopic system according to a fourth embodiment of the present invention.

FIG. 10 is a timing chart illustrating operations of the endoscopic system according to the fourth embodiment of the present invention.

FIG. 11 is a flowchart illustrating operations of an endoscopic system according to a fifth embodiment of the present invention.

### DETAILED DESCRIPTION OF THE PREFERRED EMBODIMENTS

Hereinafter, medical endoscopic systems that capture and display an image in a body cavity of a patient or the like will be described as modes for carrying out the present invention (hereinafter referred to as “embodiments”). The present invention is not limited to the embodiments. In the drawings, like elements are referenced by like reference numerals. It should be noted that the drawings are schematic and relationships between a thickness and a width of each element, ratios of elements, and the like are different from reality. Elements having different dimensions or ratios across the drawings are included.

### First Embodiment

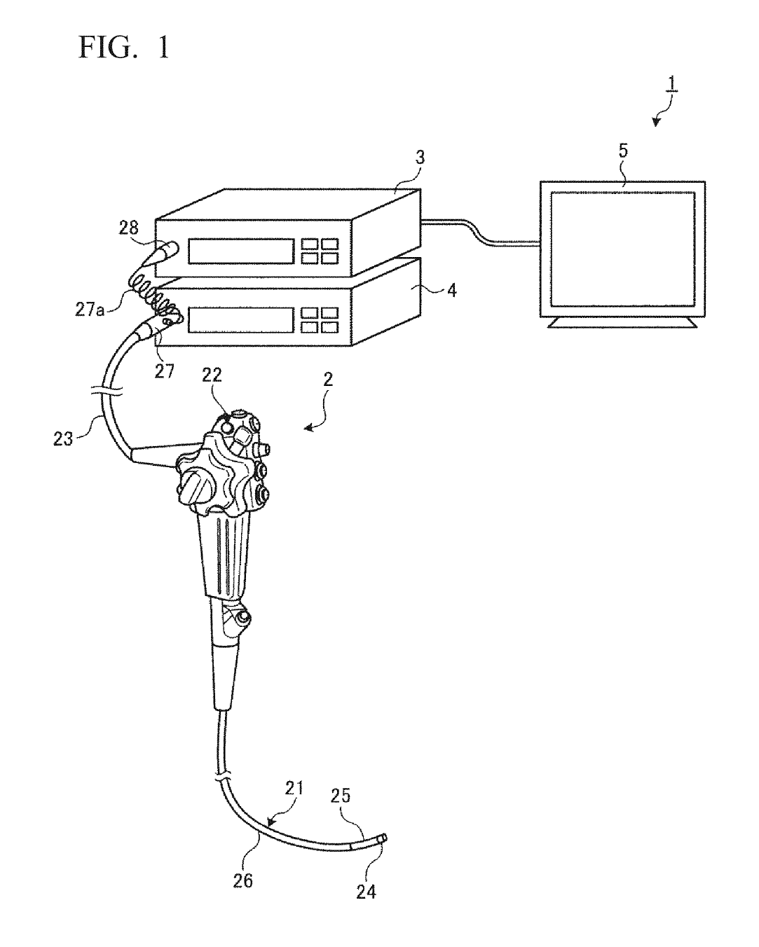

FIG. 1 is a diagram schematically illustrating a configuration of an endoscopic system according to a first embodiment of the present invention.

As illustrated in FIG. 1, an endoscopic system 1 includes an endoscope 2 (an electronic scope) that serves as an imaging device capturing an internal image of a subject by a distal end thereof being inserted into a body cavity of the subject, a processing device 3 (an external processor) that performs predetermined image processing on the captured internal image, a light source device 4 that generates illumination light which is emitted from the distal end of the endoscope 2, and a display device 5 that displays the internal image subjected to the image processing by the processing device 3.

The endoscope 2 includes an insertion section 21 that has flexibility and has a thin and long shape, an operation unit 22 that is connected to a proximal end of the insertion section 21 and receives input of various operation signals, and a universal cord 23 that extends from the operation unit 22 in a direction different from an extending direction of the insertion section 21 and has various cables for connecting to the processing device 3 built therein.

The insertion section 21 includes a distal end 24 that has an imaging element, which will be described, incorporated therein, a curving portion 25 that includes a plurality of curving pieces and can be freely curved, and a flexible tube portion 26 that is connected to a proximal end of the curving portion 25 and has a long shape having flexibility.

The operation unit 22 is operated by an operator to vertically and horizontally curve the curving portion 25.

The universal cord 23 has a cable built therein and includes a connector portion 27 that can be attached to and detached from the light source device 4. The connector portion 27 includes a coil-shaped coil cable 27a and includes a connector portion 28 that can be attached to and detached from the processing device 3 at an extension of the coil cable 27a.

The processing device 3 performs the predetermined image processing on an internal image captured by the endoscope 2 and comprehensively controls all operations of the endoscopic system 1.

The light source device 4 emits light, which is generated from a light source such as a xenon lamp or a white LED, from a tip of the distal end 24.

The display device 5 has a function of displaying an internal image generated by the processing device 3 via an image cable. The display device 5 is constituted by, for example, a liquid crystal display or an organic electroluminescence (EL) display.

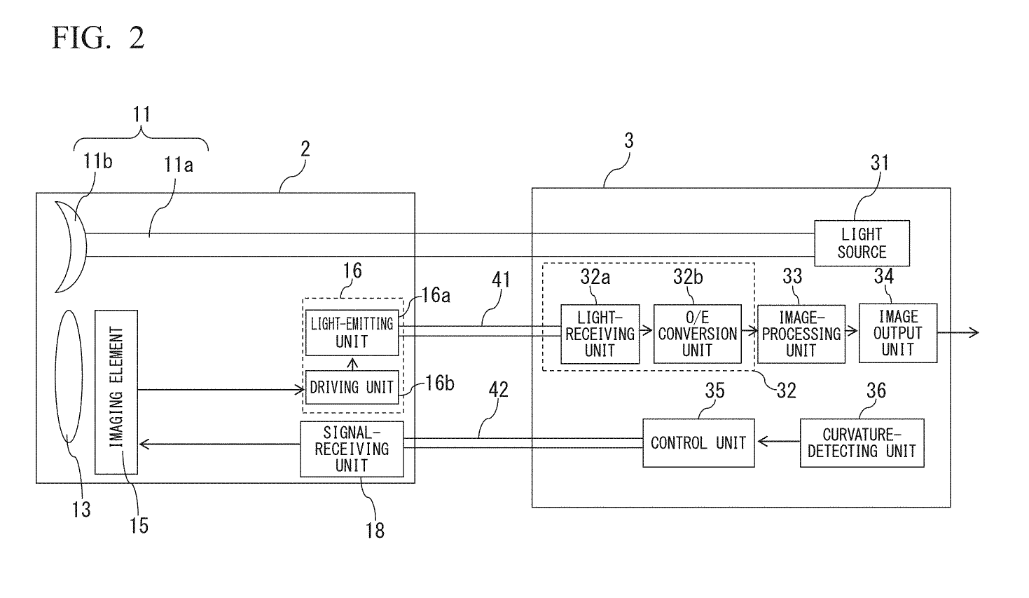

FIG. 2 is a block diagram illustrating functional configurations of principal parts of the endoscopic system according to the first embodiment of the present invention.

The endoscope 2 includes an illumination unit 11, an objective optical system 13, an imaging element 15, an optical transmission unit 16, and a signal-receiving unit 18. The processing device 3 includes a light source 31, an optical reception unit 32, an image-processing unit 33, an image output unit 34, a control unit 35, and a curvature-detecting unit 36.

The light source 31 generates illumination light which is applied to a subject. For example, a xenon lamp or a white LED is used as the light source 31.

The illumination unit 11 includes a light guide 11a and an illumination lens 11b. The illumination light generated from the light source 31 is applied to the subject via the light guide 11a and the illumination lens 11b.

The objective optical system 13 causes reflected light from the subject irradiated by the illumination unit 11 to be incident on the imaging element 15.

The imaging element 15 converts the light incident via the objective optical system 13 into an electrical signal. For example, a CCD image sensor or a CMOS image sensor can be used as the imaging element 15.

The optical transmission unit 16 includes a light-emitting unit 16a and a driving unit 16b that drives the light-emitting unit 16a, and outputs an optical signal to the processing device 3. The light-emitting unit 16a is driven by the driving unit 16b and outputs an optical signal to the processing device 3 by emitting light. The driving unit 16b drives the light-emitting unit 16a based on an output signal of the imaging element 15.

The optical reception unit 32 includes a light-receiving unit 32a and an optic/electric conversion unit (an O/E conversion unit) 32b. The light-receiving unit 32a receives the optical signal transmitted from the optical transmission unit 16. The O/E conversion unit 32b converts the optical signal received by the light-receiving unit 32a into an electrical signal and transmits the electrical signal to the image-processing unit 33.

The image-processing unit 33 performs predetermined image processing, such as gray scale correction and white balance adjustment, on the electrical signal converted by the O/E conversion unit 32b and outputs a resultant signal to the image output unit 34.

The image output unit 34 outputs an image subjected to the image processing by the image-processing unit 33 to the display device 5.

The curvature-detecting unit 36 detects a curvature of an optical fiber 41. Specifically, the curvature-detecting unit detects a shape of the optical fiber 41 using a known pressure sensor or the like (not illustrated) detecting the shape of the optical fiber 41 and detects the curvature of the optical fiber 41 based on the detected shape. The detection result of the curvature of the optical fiber 41 is output to the control unit 35.

The control unit 35 transmits a control signal to the signal-receiving unit 18 based on the detection result of the curvature-detecting unit 36. Specifically, the control unit receives the detection result on a degree of curving of the optical fiber 41 from the curvature-detecting unit 36 and transmits a control signal on characteristics of the output signal of the imaging element 15 to the signal-receiving unit 18 via a signal line 42.

The signal-receiving unit 18 transmits the control signal transmitted from the control unit 35 to the imaging element 15. The imaging element 15 adjusts the characteristics of the output signal of the imaging element 15 based on the control signal. That is, the imaging element 15 adjusts an amplitude level of the output signal of the imaging element 15 based on the control signal. An example of a method of adjusting an amplitude level of an output signal of the imaging element 15 is changing an amplification factor of an amplifier in the imaging element 15.

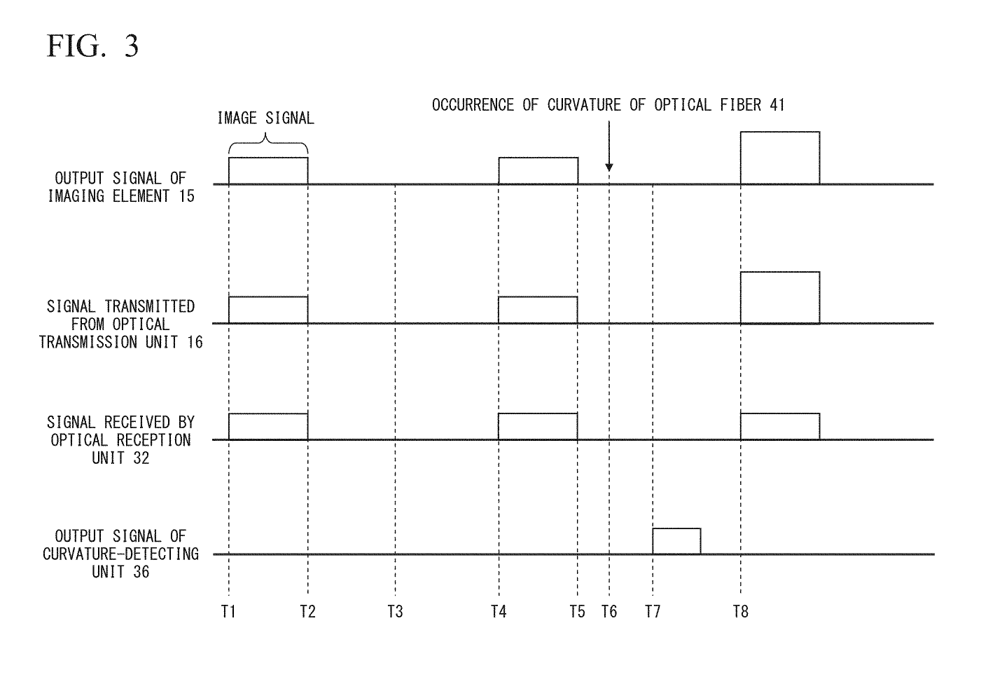

FIG. 3 is a timing chart illustrating operations of the endoscopic system according to the first embodiment of the present invention.

At timing T1, the imaging element 15 starts to output an image signal in a first frame. The optical transmission unit 16 outputs a signal converted into an optical signal based on the image signal. The optical reception unit 32 receives the optical signal output from the optical transmission unit 16. At this time, since the curving portion 25 or the like is not curved, the optical fiber 41 is not curved. Accordingly, the optical signal output from the optical transmission unit 16 is transmitted to the optical reception unit 32 via the optical fiber 41 without being attenuated.

At timing T2, the imaging element 15 ends the transmission of the image signal. At this time, the optical signal output from the optical transmission unit 16 and the optical signal received by the optical reception unit 32 are zero.

At timing T3, the curvature-detecting unit 36 detects a curvature of the optical fiber 41 and outputs the detection result to the control unit 35. Here, the curvature-detecting unit 36 outputs a high level signal to the control unit 35 when the curvature of the optical fiber 41 is detected and outputs a low level signal to the control unit 35 when the curvature of the optical fiber 41 is not detected. At timing T3, since the optical fiber 41 is not curved, the curvature-detecting unit 36 outputs the low level signal to the control unit 35. The curvature-detecting unit 36 has been described above as outputting the low level signal to the control unit 35, but the present invention is not limited thereto, and the curvature-detecting unit may transmit a signal of a predetermined pattern or may transmit a signal of predetermined amplitude.

The imaging element 15 starts to output an image signal in a second frame at timing T4 and ends the output of the image signal at timing T5.

At timing T6, the curving portion 25 or the like is curved and the optical fiber 41 is also curved. When the optical fiber 41 is curved, the optical signal output from the optical transmission unit 16 is attenuated in accordance with the curvature of the optical fiber 41. Accordingly, the optical signal output from the optical transmission unit 16 is attenuated and transmitted to the optical reception unit 32.

At timing T7, the curvature-detecting unit 36 detects the curvature of the optical fiber 41. At this time, since the optical fiber 41 is curved at timing T6, the curvature-detecting unit 36 outputs the high level signal to the control unit 35.

The control unit 35 outputs a control signal to the signal-receiving unit 18 to amplify the output signal of the imaging element 15 based on the signal output from the curvature-detecting unit 36. The imaging element 15 amplifies the output signal thereof based on the control signal.

At timing T8, the imaging element 15 starts to output an image signal in a third frame. At this time, since the imaging element 15 amplifies the output signal, an amplitude level of the output signal is higher than that in a case in which the optical fiber 41 is not curved. Since the optical transmission unit 16 outputs an optical signal converted based on the image signal, an amplitude level of the optical signal is higher than that in the case in which the optical fiber 41 is not curved.

According to the first embodiment, since the optical fiber 41 is curved, the optical signal output from the optical transmission unit 16 is attenuated. However, since the amplitude level of the optical signal is increased based on the detection result of the curvature-detecting unit 36, the optical signal can be transmitted as an optical signal of a normal amplitude level to the optical reception unit 32. That is, even when the optical fiber 41 is curved and the optical signal is attenuated, it is possible to perform appropriate optical transmission.

The control signal output from the control unit 35 may be transmitted as an optical signal or may be transmitted as an electrical signal. Alternatively, the control signal may be transmitted as a radio signal by radio communication. When the control signal is transmitted as an optical signal, an amplitude level of the optical signal is increased and the optical signal is transmitted in consideration of the curvature of the optical fiber.

An example of a signal output from the curvature-detecting unit 36 is a high level or low level binary signal, but the signal is not limited thereto. A signal level of the signal may be adjusted and the signal may be output, for example, based on the curvature of the optical fiber 41.

The curvature-detecting unit 36 is disposed in the processing device 3, but is not limited to this arrangement position, and may be disposed in the endoscope 2. In this case, the detection result of the curvature-detecting unit 36 is transmitted from the endoscope 2 to the processing device 3. The control unit 35 transmits the control signal to the signal-receiving unit 18 based on the transmitted detection result.

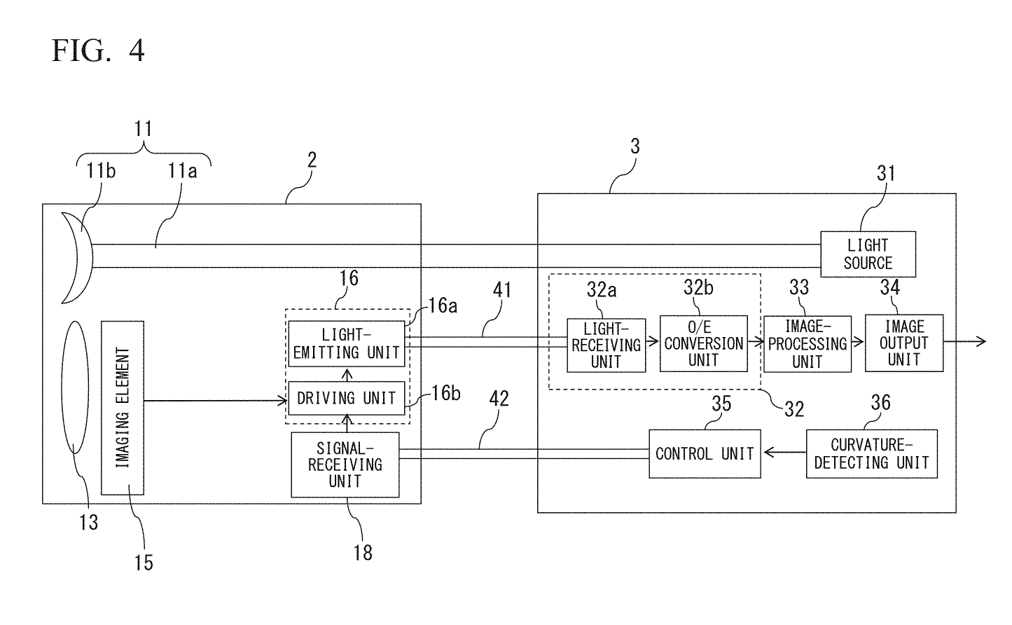

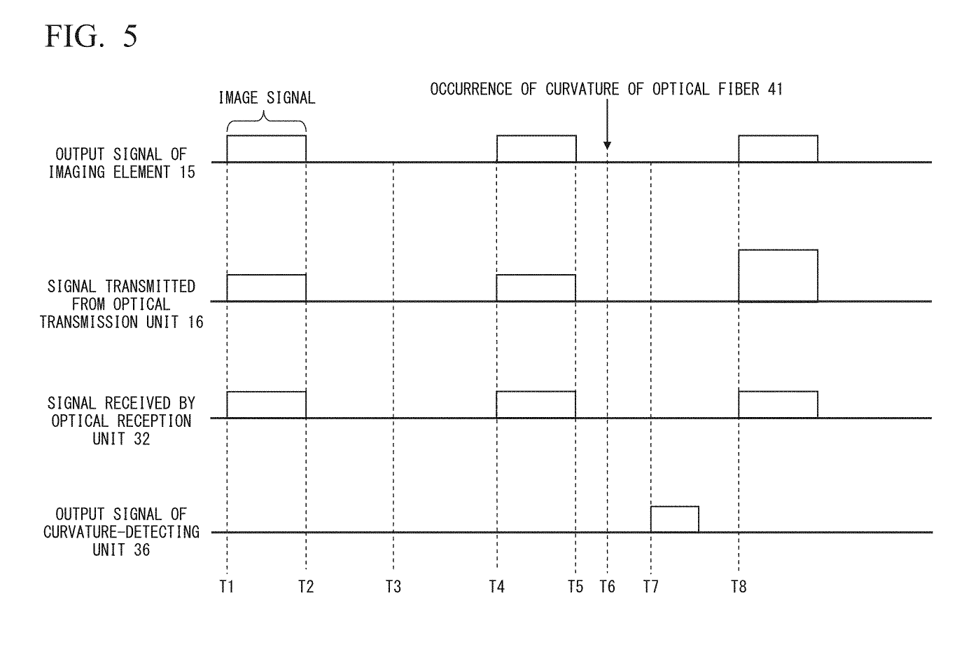

A modified example of the first embodiment will be described below with reference to FIGS. 4 and 5.

FIG. 4 is a block diagram illustrating functional configurations of principal parts of an endoscopic system according to the modified example of the first embodiment. The modified example of the first embodiment is different from the embodiment illustrated in FIG. 2 in that an output of the signal-receiving unit 18 is transmitted to the driving unit 16b.

In the modified example of the first embodiment, the control unit 35 outputs a control signal to the signal-receiving unit 18 to amplify an output signal of the light-emitting unit 16a. The signal-receiving unit 18 transmits the control signal to the driving unit 16b. The driving unit 16b amplifies the output signal of the light-emitting unit 16a based on the control signal.

FIG. 5 is a timing chart illustrating operations of the endoscopic system according to the modified example of the first embodiment. FIG. 5 is different from FIG. 3 only in timing T8. Accordingly, timing T8 will be described below.

At timing T8, the imaging element 15 outputs an image signal in a third frame. The optical transmission unit 16 amplifies and outputs an optical signal converted based on the image signal in response to a control signal transmitted from the signal-receiving unit 18. Accordingly, an amplitude level of the optical signal is higher than that in the case in which the optical fiber 41 is not curved.

According to the modified example of the first embodiment, even when the optical fiber 41 is curved and the optical signal is attenuated, the amplitude level of the optical signal is increased based on the detection result of the curvature-detecting unit 36. Accordingly, the optical signal can be transmitted as an optical signal of a normal amplitude level to the optical reception unit 32. That is, even when the optical fiber 41 is curved and the optical signal is attenuated, it is possible to perform appropriate optical transmission.

### Second Embodiment

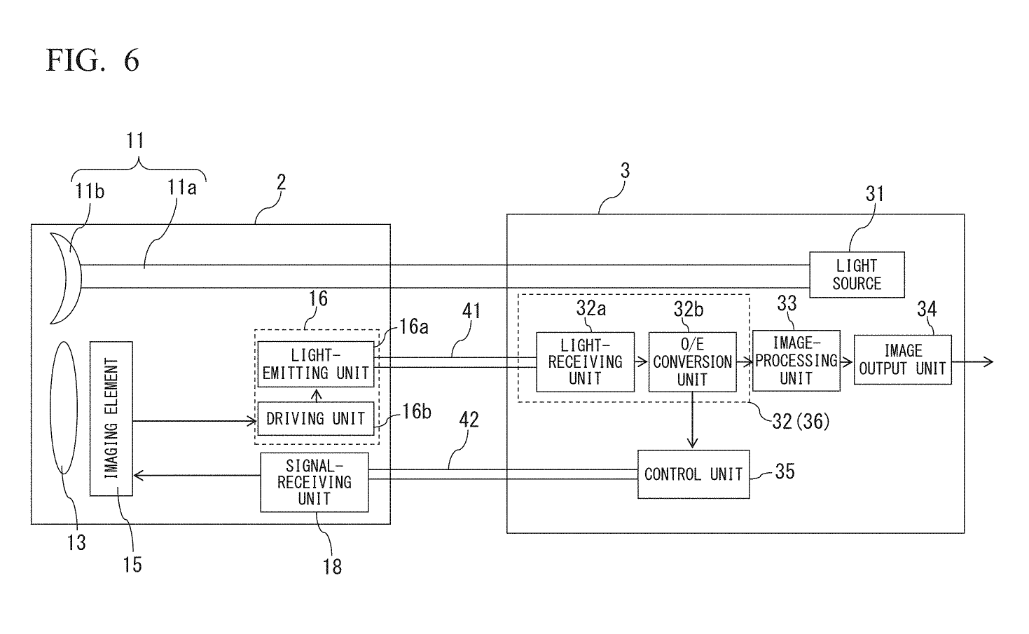

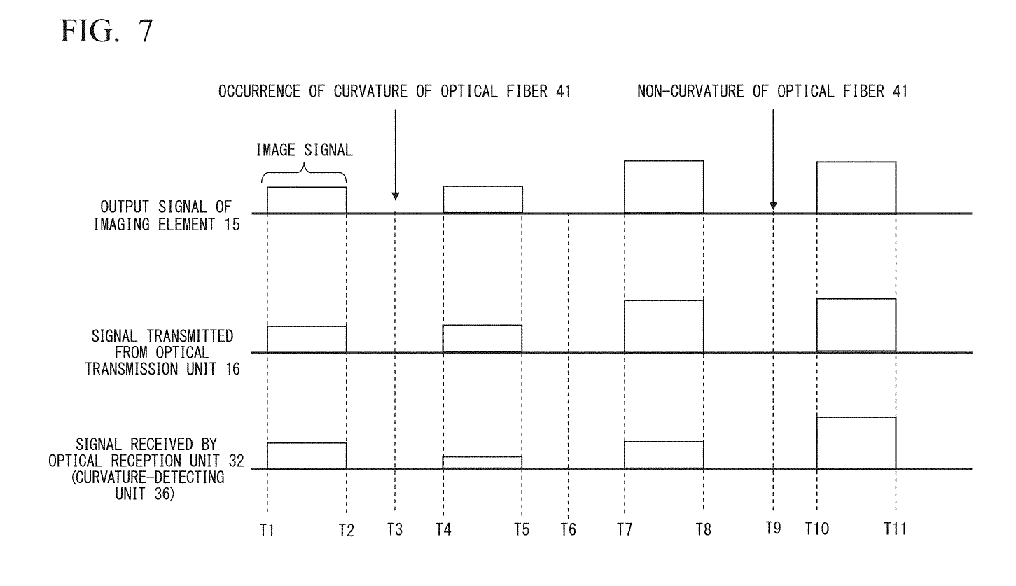

A second embodiment will be described below with reference to FIGS. 6 and 7.

In the first embodiment, the curvature-detecting unit 36 detects the curvature of the optical fiber 41 using a sensor that detects the shape of the optical fiber 41, but the second embodiment is different from the first embodiment in that the optical reception unit 32 detects the curvature of the optical fiber 41 based on the received optical signal. That is, both embodiments are different from each other in that the optical reception unit 32 also serves as the curvature-detecting unit 36.

FIG. 6 is a block diagram illustrating functional configurations of principal parts of an endoscopic system according to the second embodiment.

A light-receiving unit 32a receives an optical signal output from a light-emitting unit 16a and outputs the received optical signal to an O/E conversion unit 32b. The O/E conversion unit 32b converts the optical signal output from the light-receiving unit 32a into an electrical signal and outputs the electrical signal to an image-processing unit 33 and a control unit 35.

The control unit 35 transmits a control signal to a signal-receiving unit 18 based on the electrical signal converted by the O/E conversion unit 32b. Specifically, a control signal relevant to characteristics of an output signal of an imaging element 15 is transmitted to the signal-receiving unit 18 via a signal line 42 using the electrical signal converted by the O/E conversion unit 32b.

The signal-receiving unit 18 outputs the control signal to the imaging element 15, and the imaging element 15 amplifies the output signal thereof based on the control signal.

FIG. 7 is a timing chart illustrating operations of the endoscopic system according to the second embodiment.

At timing T1, the imaging element 15 starts to output an image signal in a first frame. At timing T2, the imaging element 15 stops outputting the image signal in the first frame.

At timing T3, the optical fiber 41 is curved.

The imaging element 15 starts to output an image signal in a second frame at timing T4 and stops outputting the image signal in the second frame at timing T5. At this time, since the optical fiber 41 is curved at timing T3, the signal transmitted from the optical transmission unit 16 is attenuated due to light leakage. Accordingly, an amplitude level of the signal received by the optical reception unit 32 (the curvature-detecting unit 36) is lower than that in a case in which the optical fiber 41 is not curved.

At timing T6, the control unit 35 outputs a control signal to the signal-receiving unit 18 to amplify the output signal of the imaging element 15 based on the amplitude level of the signal transmitted from the optical reception unit 32 (the curvature-detecting unit 36). The imaging element 15 amplifies the output signal thereof based on the control signal.

The imaging element 15 starts to output an image signal in a third frame at timing T7 and stops outputting the image signal in the third frame at timing T8. At this time, since the imaging element 15 amplifies the output signal, the amplitude level of the output signal is higher than that in the case in which the optical fiber 41 is not curved. Since the optical transmission unit 16 outputs the optical signal converted based on the image signal, an amplitude level of the optical signal is higher than that in the case in which the optical fiber 41 is not curved.

At timing T9, the curvature of the optical fiber 41 is released.

The imaging element 15 starts to output an image signal in a fourth frame at timing T10 and stops outputting the image signal in the fourth frame at timing T11. At this time, the imaging element 15 amplifies the output signal. Accordingly, the amplitude level of the optical signal received by the optical reception unit 32 is very high. At this time, the control unit 35 determines that the curvature of the optical fiber 41 is released and outputs a control signal to the signal-receiving unit 18 to release a process of amplifying the output signal of the imaging element 15.

When the optical fiber 41 is curved after stopping the output of the output signal in the fourth frame from the imaging element 15 (after timing T11), the same processes as from timing T4 to timing T8 are performed.

Like in the modified example of the first embodiment, the output signal of the light-emitting unit 16a may be amplified instead of amplifying the output signal of the imaging element 15. The control unit 35 may detect light intensity of the light-receiving unit 32a and amplify the output signal of the imaging element 15 or the light-emitting unit 16a.

According to the second embodiment, the optical signal output from the optical transmission unit 16 is attenuated because the optical fiber 41 is curved. However, since the amplitude level of the optical signal is increased based on the output signal of the optical reception unit 32, the optical signal can be transmitted as an optical signal of a normal amplitude level to the optical reception unit 32. That is, even when the optical fiber 41 is curved and the optical signal is attenuated, it is possible to perform appropriate optical transmission. Since the optical reception unit 32 also serves as the curvature-detecting unit 36, it is possible to achieve a decrease in cost and to achieve a decrease in size of the endoscopic system.

### Third Embodiment

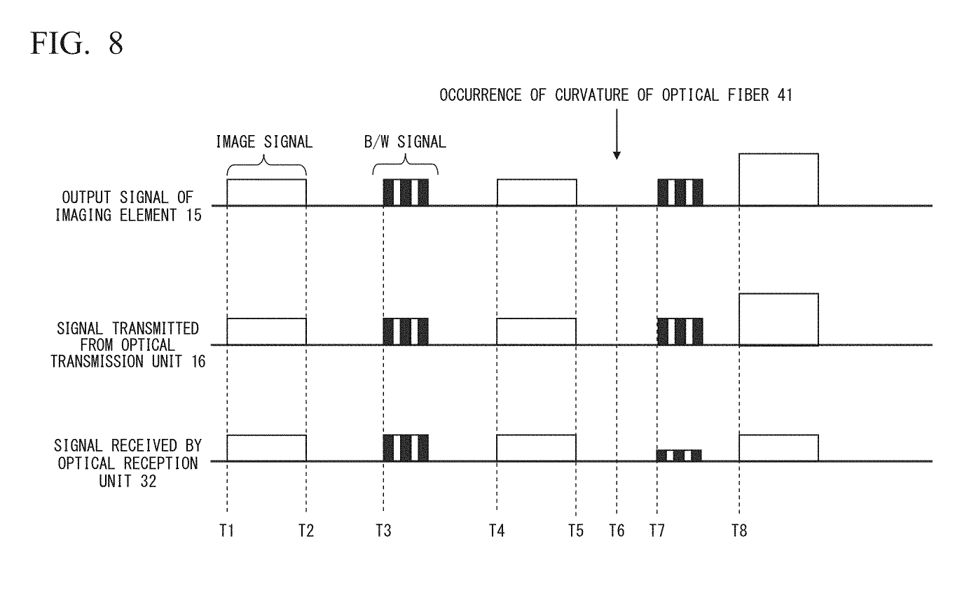

A third embodiment will be described below with reference to FIG. 8. A block diagram illustrating functional configurations of principal parts of an endoscopic system according to the third embodiment is the same as in the second embodiment illustrated in FIG. 6 and will not be illustrated.

An imaging element 15 according to the third embodiment outputs a predetermined electrical signal in addition to a captured image signal. An example of the predetermined electrical signal is a black/white signal (a B/W signal). The B/W signal is an alternating output of an electrical signal when a black image is captured and an electrical signal when a white image is captured. A signal of an optical black pixel may be used as the predetermined electrical signal.

FIG. 8 is a timing chart illustrating operations of the endoscopic system according to the third embodiment.

The imaging element 15 alternately outputs a captured image signal and a predetermined electrical signal. Specifically, at timing T1, the imaging element 15 starts to output an image signal in a first frame. An optical transmission unit 16 outputs a signal which has been converted into an optical signal based on the image signal in the first frame. An optical reception unit 32 receives the optical signal output from the optical transmission unit 16. At this time, since a curving portion 25 or the like is not curved, an optical fiber 41 is not curved. Accordingly, the optical signal output from the optical transmission unit 16 is transmitted to an optical reception unit 32 via the optical fiber 41 without being attenuated.

At timing T2, the imaging element 15 stops transmitting the image signal. At this time, the optical signal output from the optical transmission unit 16 and the optical signal received by the optical reception unit 32 are zero.

At timing T3, the imaging element 15 outputs a predetermined electrical signal. The predetermined electrical signal is used to detect whether transmission of the optical signal between the optical transmission unit 16 and the optical reception unit 32 is normally performed. The predetermined electrical signal is output, for example, in a horizontal blanking period or a vertical blanking period.

Since the optical fiber 41 is not curved, the optical signal output from the optical transmission unit 16 based on the predetermined electrical signal output from the imaging element 15 is transmitted to the optical reception unit 32 without being attenuated.

The imaging element 15 starts to output an image signal in a second frame at timing T4 and stops outputting the image signal at timing T5.

At timing T6, the curving portion 25 or the like is curved and the optical fiber 41 is also curved. When the optical fiber 41 is curved, the optical signal output from the optical transmission unit 16 is attenuated in accordance with the curvature of the optical fiber 41. Accordingly, the optical signal output from the optical transmission unit 16 is attenuated and transmitted to the optical reception unit 32.

At timing T7, the imaging element 15 outputs the predetermined electrical signal again. The optical transmission unit 16 outputs an optical signal converted based on the predetermined electrical signal. At this time, the optical signal corresponding to the predetermined electrical signal is attenuated and transmitted to the optical reception unit 32 due to the curvature of the optical fiber 41.

The attenuated and transmitted optical signal is converted into an electrical signal by an O/E conversion unit 32b. A control unit 35 detects an amplitude level of the electrical signal converted by the O/E conversion unit 32b. At this time, since the amplitude level of the electrical signal converted by the O/E conversion unit 32b is very low, the control unit 35 outputs a control signal to the signal-receiving unit 18 to amplify the output signal of the imaging element 15. The imaging element 15 amplifies the output signal thereof based on the control signal.

For example, when the amplitude level of the electrical signal converted by the O/E conversion unit 32b is 1/γ (γ>1) times that in a state in which the optical fiber 41 is not curved, the control unit 35 controls an amplitude level of the output signal of the imaging element 15 to be multiplied by γ.

At timing T8, the imaging element 15 starts to output an image signal in a third frame. At this time, since the imaging element 15 amplifies the output signal, the amplitude level of the output signal is higher than that in the case in which the optical fiber 41 is not curved. Since the optical transmission unit 16 outputs an optical signal converted based on the image signal, an amplitude level of the optical signal is higher than that in the case in which the optical fiber 41 is not curved.

According to the third embodiment, since the optical fiber 41 is curved, the optical signal output from the optical transmission unit 16 is attenuated. However, since the amplitude level of the optical signal is amplified based on the predetermined electrical signal output from the imaging element 15, the optical signal can be transmitted as an optical signal of a normal amplitude level to the optical reception unit 32. That is, even when the optical fiber 41 is curved and the optical signal is attenuated, it is possible to perform normal optical transmission. Even when the optical fiber 41 is curved in an imaging frame, it is possible to perform appropriate optical transmission in a next frame.

### Fourth Embodiment

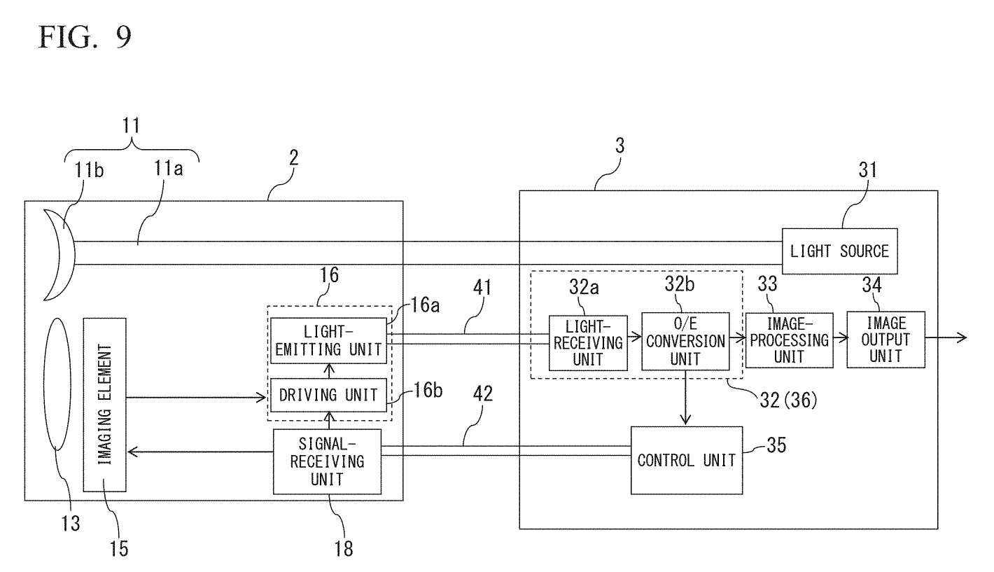

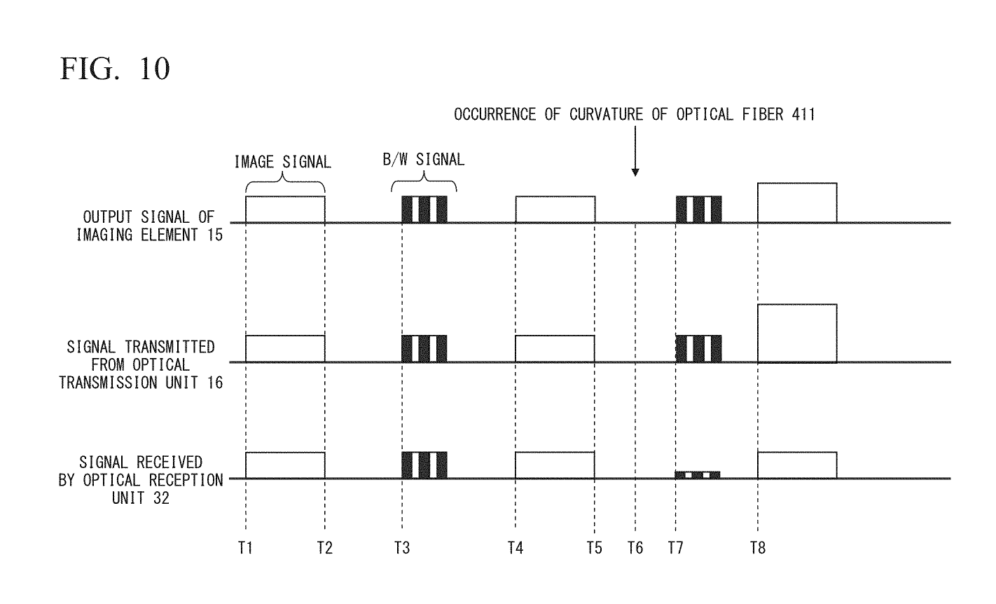

A fourth embodiment will be described below with reference to FIGS. 9 and 10.

In the third embodiment, the amplitude level of the output signal of the imaging element 15 is controlled based on the control signal output from the control unit 35. However, in the fourth embodiment, output characteristics of an imaging element 15 and an optical transmission unit 16 are controlled based on a control signal output from a control unit 35. That is, amplitude levels of the output signals of both the imaging element 15 and the optical transmission unit 16 are controlled based on the control signal output from the control unit 35.

FIG. 9 is a block diagram illustrating functional configurations of principal parts of an endoscopic system according to the fourth embodiment of the present invention.

The control unit 35 outputs a control signal to a signal-receiving unit 18 based on an amplitude level of an electrical signal converted by an O/E conversion unit 32b.

The signal-receiving unit 18 transmits the control signal to the imaging element 15 and the optical transmission unit 16.

The imaging element 15 adjusts the amplitude level of the output signal thereof based on the control signal transmitted from the signal-receiving unit 18. The optical transmission unit 16 adjusts the amplitude level of the output signal of the optical transmission unit 16 based on the control signal transmitted from the signal-receiving unit 18.

FIG. 10 is a timing chart illustrating operations of the endoscopic system according to the fourth embodiment of the present invention. The processes up to timing T7 are the same as described above with reference to FIG. 8 and description thereof will not be repeated.

At timing T7, the imaging element 15 outputs a predetermined electrical signal. The optical transmission unit 16 outputs an optical signal converted based on the predetermined electrical signal. At this time, the optical signal is attenuated and transmitted to an optical reception unit 32 due to a curvature of an optical fiber 41.

The attenuated and transmitted optical signal is converted into an electrical signal by the O/E conversion unit 32b. The control unit 35 detects an amplitude level of the electrical signal converted by the O/E conversion unit 32b. At this time, since the amplitude level of the electrical signal converted by the O/E conversion unit 32b is very low, the control unit 35 outputs a control signal to the signal-receiving unit 18 to amplify the output signal of the imaging element 15.

For example, when the amplitude level of the electrical signal converted by the O/E conversion unit 32b is 1/γ times that in a state in which the optical fiber 41 is not curved, the control unit 35 controls the amplitude level of the output signal of the imaging element 15 to be multiplied by α₁ and controls the amplitude level of the output signal of the optical transmission unit 16 to be multiplied by β₁. Here, a relationship of α₁ and β₁ is set to satisfy γ=α₁×β₁ (where α₁>1 and β₁>1).

The imaging element 15 and the optical transmission unit 16 are set to amplify the output signals thereof based on the control signal.

At timing T8, the imaging element 15 increases the amplitude level of the output signal thereof based on the control signal and outputs the output signal.

The optical transmission unit 16 outputs an optical signal, which is obtained by additionally amplifying the signal amplified by the imaging element 15, to the optical reception unit 32.

In the fourth embodiment, the optical transmission unit 16 additionally amplifies the signal amplified by the imaging element 15 and outputs the amplified signal. Accordingly, even when the curvature of the optical fiber 41 is very large, it is possible to normally transmit a signal. Since an amplitude level of a signal to be transmitted is adjusted by both the imaging element 15 and the optical transmission unit 16, it is possible to finely set the amplitude level of the signal to be transmitted.

### Fifth Embodiment

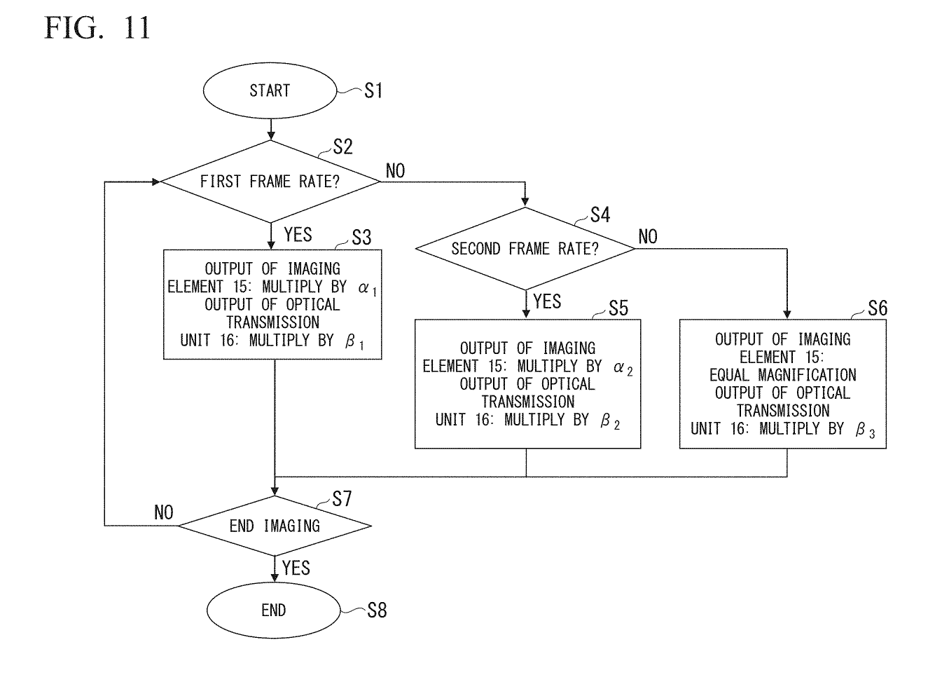

A fifth embodiment will be described below with reference to FIG. 11.

Functional configurations of principal parts of an endoscopic system according to the fifth embodiment are the same as in the fourth embodiment, except that an imaging element 15 has a plurality of imaging frame rates (also simply referred to as frame rates). Accordingly, description of the functional configurations of the principal parts of the endoscopic system according to the fifth embodiment will not be repeated.

The imaging element 15 has modes in which imaging is performed at a first frame rate, imaging is performed at a second frame rate higher than the first frame rate, and imaging is performed at a third frame rate higher than the second frame rate.

For example, 30 fps is assumed to be set as the first frame rate, 60 fps is assumed to be set as the second frame rate, and 120 fps or 240 fps is assumed to be set as the third frame rate. The embodiment is not limited to the frame rates as long as the second frame rate is higher than the first frame rate and the third frame rate is higher than the second frame rate. A mode can be switched to increase a frame rate when an endoscope moves quickly and to decrease the frame rate when the endoscope moves slowly.

FIG. 11 is a flowchart illustrating operations of an endoscopic system according to the fifth embodiment of the present invention.

In Step S1, imaging is started by an endoscopic system 1. For the purpose of simplification of explanation, it is assumed that an optical fiber 41 is greatly curved at this time.

In Step S2, a control unit 35 determines whether a frame rate of an imaging element 15 is the first frame rate. A process of Step S3 is performed when the control unit 35 determines that the frame rate of the imaging element 15 is the first frame rate, and a process of Step S4 is performed when the control unit determines that the frame rate of the imaging element 15 is not the first frame rate.

In Step S3, the control unit 35 adjusts an amplitude level of an output signal of the imaging element 15 and an amplitude level of an output signal of an optical transmission unit 16. That is, the control unit 35 outputs a controls signal to a signal-receiving unit 18 to amplify the output signal of the imaging element 15 and the output signal of the optical transmission unit 16.

Specifically, the amplitude level of the output signal of the imaging element 15 is increased by α₁ times and the amplitude level of the output signal of the optical transmission unit 16 is increased by β₁ times. Here, similarly to the first embodiment, γ=α₁×β₁ (where α₁>1 and β₁>1) is set to be satisfied when an amplitude level of an optical signal received by an optical reception unit 32 is 1/γ times that in a state in which the optical fiber 41 is not curved.

In Step S4, the control unit 35 determines whether the frame rate of the imaging element 15 is the second frame rate. A process of Step S5 is performed when the control unit 35 determines that the frame rate of the imaging element 15 is the second frame rate, and a process of Step S6 is performed when the control unit determines that the frame rate of the imaging element 15 is not the second frame.

In Step S5, the control unit 35 adjusts the amplitude level of the output signal of the imaging element 15 and the amplitude level of the output signal of the optical transmission unit 16. That is, the control unit 35 outputs a controls signal to the signal-receiving unit 18 to amplify the output signal of the imaging element 15 and the output signal of the optical transmission unit 16.

Specifically, the amplitude level of the output signal of the imaging element 15 is increased by α₂ times and the amplitude level of the output signal of the optical transmission unit 16 is increased by β₂ times. Here, similarly to the first embodiment, γ=α₂×β₂ (where α₂>1 and β₂>1) and α₁>α₂ and β₁<β₂ are set to be satisfied when the amplitude level of the optical signal received by the optical reception unit 32 is 1/γ times that in the state in which the optical fiber 41 is not curved.

That is, in comparison with the case in which the frame rate is the first frame rate, a ratio at which the amplitude level of the output signal of the imaging element 15 is adjusted is set to be smaller and a ratio at which the amplitude level of the output signal of the optical transmission unit 16 is adjusted is set to be greater.

In Step S6, since the frame is not the first frame rate and is not also the second frame rate, the control unit 35 determines that the frame rate of the imaging element 15 is the third frame rate.

At this time, the control unit 35 sets the amplitude level of the output signal of the imaging element 15 to be an equal magnification. That is, the control unit 35 sets the amplitude level of the output signal of the imaging element 15 to be the same amplitude level as in the case in which the optical fiber 41 is not curved. In other words, the control unit 35 stops adjustment of the amplitude level of the output signal of the imaging element 15.

On the other hand, the control unit 35 sets the output of the optical transmission unit 16 to be multiplied by β₃ (β₃>1). Here, similarly to the first embodiment, γ=β₃ and β₂<β₃ are set to be satisfied when the amplitude level of the optical signal received by the optical reception unit 32 is 1/γ times that in the state in which the optical fiber 41 is not curved.

In Step S7, it is determined whether the imaging by the endoscopic system 1 has ended. When the imaging has not ended, the process of Step S2 is performed again. When the imaging has ended, the process of Step S8 is performed.

In the fifth embodiment, the control unit 35 determines the frame rate of the imaging element 15 and changes adjustment ratios of the amplitude level of the output signal of the imaging element 15 and the amplitude level of the output signal of the optical transmission unit 16. When the frame rate of the imaging element 15 is high and the adjustment ratio of the amplitude level of the output signal of the imaging element 15 is high, power consumption of the imaging element 15 increases and an amount of heat emitted therefrom increases. However, in this embodiment, when the frame rate of the imaging element 15 is high, the adjustment ratio of the amplitude level of the output signal of the optical transmission unit 16 is set to be high and it is possible to suppress an increase in power consumption of the imaging element 15 and to suppress an amount of heat emitted therefrom.

When the frame rate of the imaging element 15 is very high, the adjustment of the amplitude level of the output signal of the imaging element 15 is stopped and only the amplitude level of the output signal of the optical transmission unit 16 is adjusted. Accordingly, even when the frame rate of the imaging element 15 is very high, it is possible to suppress an amount of heat emitted from the imaging element 15.

While embodiments of the present invention have been described above in detail with reference to the drawings, the specific configurations are not limited to the embodiments but include changes in design that do not depart from the gist of the present invention. Medical endoscopic systems have been exemplified in the embodiments of the present invention, but the present invention is not limited to the medical endoscopic systems, and can also be applied to industrial endoscopic systems.

What is claimed is:

1. An endoscopic system comprising:
an endoscope configured to acquire an image of an inside of a subject; and
a processing device configured to perform an image processing on the acquired image,
wherein the endoscope includes:
an imaging unit configured to output the image of the inside of the subject as an electrical signal; and
an optical transmission unit configured to convert the electrical signal into an optical signal and transmit the optical signal to the processing device via an optical fiber,
the processing device includes:
an optical reception unit configured to receive the optical signal transmitted from the optical transmission unit and convert the received optical signal into an electrical signal;
a curvature-detecting unit configured to perform a detection of a curvature of the optical fiber; and
a control unit configured to control characteristics of the electrical signal output from the imaging unit based on a result of the detection by the curvature-detecting unit,
the imaging unit includes an amplifier configured to amplify the electrical signal to be output from the imaging unit, and
the control unit is configured to control the characteristics of the electrical signal output from the imaging unit by changing an amplification factor of the amplifier.

2. The endoscopic system according to claim 1, wherein the curvature-detecting unit is configured to detect the curvature of the optical fiber based on the optical signal received by the optical reception unit.

3. The endoscopic system according to claim 2, wherein the curvature-detecting unit is configured to detect the curvature of the optical fiber based on an amplitude of the optical signal received by the optical reception unit.

4. The endoscopic system according to claim 2, wherein the curvature-detecting unit is configured to convert the optical signal received by the optical reception unit into an electrical signal and detect the curvature of the optical fiber based on the converted electrical signal.

5. The endoscopic system according to claim 1, wherein the control unit is configured to detect an imaging frame rate of the imaging unit and change an adjustment ratio of the characteristics of the electrical signal output from the imaging unit and characteristics of the optical signal output from the optical transmission unit based on the imaging frame rate.

6. The endoscopic system according to claim 5, wherein the control unit is configured to stop controlling the characteristics of the electrical signal output from the imaging unit and control the characteristics of the optical signal output from the optical transmission unit when the imaging frame rate is equal to or greater than a predetermined value.

7. The endoscopic system according to claim 1, wherein the imaging unit is configured to output a predetermined electrical signal other than the electrical signal of the image of the inside of the subject in a horizontal blanking period or a vertical blanking period, and
the curvature-detecting unit is configured to detect the curvature of the optical fiber based on an amplitude of the predetermined electrical signal.

8. An endoscopic system comprising:
an endoscope configured to acquire an image of an inside of a subject; and
a processing device configured to perform an image processing on the acquired image,
wherein the endoscope includes:
an imaging unit configured to output the image of the inside of the subject as an electrical signal;
an optical transmission unit configured to convert the electrical signal into an optical signal and transmit the optical signal to the processing device via an optical fiber; and
a curvature-detecting unit configured to detect a curvature of the optical fiber,
the processing device includes:
an optical reception unit configured to receive the optical signal transmitted from the optical transmission unit and convert the received optical signal into an electrical signal; and
a control unit configured to control at least one of characteristics of the electrical signal output from the imaging unit and characteristics of the optical signal output from the optical transmission unit based on the detection result of the curvature-detecting unit,
the imaging unit includes an amplifier configured to amplify the electrical signal to be output from the imaging unit, and
the control unit configured to control the characteristics of the electrical signal output from the imaging unit by changing an amplification factor of the amplifier.

9. An endoscope comprising:
an imaging unit configured to output an image of an inside of a subject as an electrical signal;
an optical transmission unit configured to convert the electrical signal into an optical signal and transmit the optical signal to the outside via an optical fiber; and
a signal-receiving unit configured to receive a control signal on characteristics of the electrical signal output from the imaging unit based on a curvature of the optical fiber,
wherein the imaging unit includes an amplifier configured to amplify the electrical signal, and
the amplifier is configured to adjust the characteristics of the electrical signal output from the imaging unit by changing an amplification factor thereof based on the control signal.
